(19) United States
(12) Patent Application Publication
(10) Pub. No.: US 2021/0299015 A1
(43) Pub. Date: Sep. 30, 2021

(54) WATER-CONTAINING LIPSTICK AND PREPARATION METHOD THEREFOR

(71) Applicant: GLOBAL COSMETICS (HONG KONG) COMPANY LIMITED, Kowloon, Hong Kong (CN)

(72) Inventors: Chun Hua ZHANG, Kowloon, Hong Kong (CN); Lin HE, Kowloon, Hong Kong (CN); Hin Yiu LAU, Kowloon, Hong Kong (CN); Yuet Wah LAU, Kowloon, Hong Kong (CN)

(21) Appl. No.: 17/264,683

(22) PCT Filed: Jul. 25, 2019
(86) PCT No.: PCT/IB2019/056349

§ 371 Date: Jan. 29, 2021

(30) Foreign Application Priority Data
Aug. 2, 2018 (CN) 201810873287.7

(51) Int. Cl. A61K 8/37; A61K 8/92; A61Q 1/06; A61K 8/02; A61K 8/06

(52) U.S. Cl. CPC A61K 8/375; A61K 8/922; A61Q 1/06; A61K 8/927; A61K 2800/524; A61K 8/06; A61K 2800/43; A61K 2800/805; A61K 8/0229

(57) ABSTRACT

The invention provides a water-containing lipstick and preparation method therefor, said water-containing lipstick comprises the following components in terms of weight percentage: Emollient 20-40%, Hardener 13-15%, Brightener 10-15%, Polyglyceryl-2-Triisostearate 10-15%, Polyglyceryl-2-Diisostearate 10-15%, Pigment 0.5-5%, Lake 0.5%-5% and Humectant 10%. The water-containing lipstick of the invention has high level of water content which can reach 10%, can add different kinds of water soluble ingredients, has high stability, dropping point can reach 56-60° C., excellent effect of emulsification, no ultrasonic equipment is required to eliminate bubbles when filling the mould (can use general filling equipment). This lipstick has various colors and high saturation, it can be easily distinguished after using on the lips.

### FIELD OF THE INVENTION

The invention belongs to the field of cosmetic, it relates to a water-containing lipstick and the preparation method of making.

### BACKGROUND OF THE INVENTION

Lipstick is a product which makes the lips ruddy, lustrous and moist, protects the lips and increase beauty of the face from modifying contour. It is ladies' essential beauty product. The regular formulas are Grease, Wax, Softener, Coloring Agent, Antioxidant, Spice and etc. Emulsifying formula comprises relatively low Water content, Lake and Pigment.

CN 107669518A disclosed a lipstick, comprising Fiber, Grease, Preservative and Auxiliary Additive. Wherein, the part of Water content by weight is 62-69, part of Inorganic Filler by weight is 6-9, part of Pigment by weight is 0.3-1, part of Thickening Agent by weight is 4.06-5.5, part of Grease by weight is 2-3, part of Emulsifier by weight is 1.5-4.5, part of Humectant by weight is 8-13, part of Preservative by weight is 0.68-0.85, part of Auxiliary Additive by weight is 0.55-1. This lipstick comprises plenty of water contents, it's wax-free, lightweight, non-sticky, fresh and cool, bright-colored, enduring against water and sweat. However, the stability of this lipstick is relatively weak, its dropping point is low.

CN 107744498A disclosed the formula of a moisturizing lipstick, said formula is calculated by percentage, comprising the following components: Olive Oil 15-28%, Jojoba Oil 8-16%, Avocado Oil 5-14%, Shea Butter 5-10%, Tea Tree Oil 3-7%, Candelilia Wax 10-20%, Bees Wax 5-15%, Glyceryl Monostearate 6-10%, Vitamin E 0.2-0.7%, Hyaluronic Acid 1-3%, Glycerol 3-7%, Honeysuckle Extract 0.15-0.35%, Sophora flavescens Extract 0.15-0.35%, Paeonia lactiflora Pall Extract 0.15-0.35%, Daucus Carrot Extract 1-3%, Carmine 0.8-2.2%. This lipstick is non-irritating to lips and skin due to all components being natural-occurring substances, it can increase the level of lips' moisturizing to against inflammation; and safety can be ensured even it gets into the mouth carelessly. However, the formula of this lipstick is too complicated, water content is low and stability is relatively poor.

CN104606093A disclosed an emulsifying lipstick, comprising the following components: water, 2-Octyl-1-Dodecanol, Pentaerythityl Tetraisostearate, Decamethylcyclopentasiloxane, Ethyl Cellulose, Etanol, Glycerol, Polyglyceryl-2 Triisostearate, Ethylene/Propylene/Styrene Copolymer, Butene/Ethylene/Styrene Copolymer, Isododecane, Polyglyceryl-6 Polyricinoleate, Trimethylolpropane Triisooctadecanoate, Coloring Agent, Disteardimonium Hectorite, Squalane Oil, Polyglyceryl-2 Isostearate, Chondrus crispus, Sodium Chloride, 2-Phenoxyethanol and Ethylhexylglycerin. This lipstick has relatively high level of gloss, color saturation and moisture, it can last long and well with makeup. Besides, it also covers the cheilogramma effectively but not give the greasy feeling. However, the water content of this emulsifying lipstick is not high. Residual value is about 50% in four hours after using it; stability is not high.

Now the lipstick which has high level of water content (various water soluble nutritional components can be added), high coloring (difference between appearance and applying color is very big) and high stability is lacked in the market. It would be a significant revolution to lipstick formulation system if such lipstick can be developed and the complicated production process can be omitted.

### SUMMARY OF THE INVENTION

This invention aims to provide a water-containing lipstick (has high level of water content and good stability) and its preparation method of making that simplify production process. In order to achieve it, the invention uses following art scheme:

First, the invention provides a water-containing lipstick, said water-containing lipstick comprises the following components in terms of weight percentage: Emollient 20-40%, Hardener 13-15%, Brightener 10-15%, Polyglyceryl-2-Triisostearate 10-15%, Polyglyceryl-2-Diisostearate 10-15%, Pigment 0.5-5%, Lake 0.5%-5% and Humectant 10%.

The water-containing lipstick provided in the invention has high level of water content which can reach 10%, can add different kinds of water soluble ingredients, has high stability, dropping point can reach 56-60° C., excellent effect of emulsification, no ultrasonic equipment is required to eliminate bubbles when filling the mould (can use general filling equipment). This lipstick has various colors and high saturation, it can be easily distinguished after using on the lips.

The water-containing lipstick provided in the invention, its water content can reach 10%. This is very advantageous to add the ingredients with various functions; the lipstick can not only eliminate dryness, moisten the lips, but also have outstanding effect in protecting lips.

The invention takes effect through Polyglyceryl-2-Triisostearate coordinating with Polyglyceryl-2-Diisostearate. It will lower the stability of lipstick and influence its taste if any one is missing.

In the invention, the weight percentage of said Emollient is 20-40%, for example, it can be 20%, 22%, 25%, 28%, 30%, 32%, 35%, 38% or 40%, etc.

Preferably, said Emollient comprises any one component or the combination of at least two components of Polyoxyethylene Glyceryl Ether Cocoate, Ethylhexyl Palmitate, Olea europaea (Olive) Fruit Oil, Simmondsia chinensis (Jojoba) Seed Oil, Coconut Oil, Cocoamidopropyl Betaine, Octyl Methoxycinnamate, Meadowfoam Seed Oil, Dipolyhydroxystearate, Octyl 4-Methoxycinnamate or Avobenzone. Said Emollient can be used individually or collaboratively.

For example, it can be the combination of Squalane Oil, Polyoxyethylene Glyceryl Ether Cocoate, Ethylhexyl Palmitate, Olea europaea (Olive) Fruit Oil, Simmondsia chinensis (Jojoba) Seed Oil and Coconut Oil; or the combination of Ethylhexyl Palmitate, Octyl 4-Methoxycinnamate and Avobenzone.

In the invention, the weight percentage of said Hardener is 13-15%, for example, it can be 13%, 13.5%, 14%, 14.5%, or 15%, etc.

Preferably, said Hardener comprises any one component or the combination of at least two components of Bees Wax, Polyethylene, Carnauba Wax or Methyl Cellulose.

In the invention, the weight percentage of said Brightener is 10-15%, for example, it can be 10%, 11%, 12%, 13%, 14% or 15%, etc.

Preferably, said Brightener comprises any one component or the combination of at least two components of Cetyl Ethylhexanoate, Squalane Oil or Tri(Tridecyl) Benzene-1,2,4-Tricarboxylate.

In the invention, Brightener can make the lipstick color more gorgeous distinguished and highlighted.

In the invention, the weight percentage of said Polyglyceryl-2-Triisostearate is 10-15%, for example, it can be 10%, 11%, 12%, 13%, 14% or 15%, etc.

In the invention, the weight percentage of said Polyglyceryl-2-Diisostearate is 10-15%, for example, it can be 10%, 11%, 12%, 13%, 14% or 15%, etc.

In the invention, the use of Polyglyceryl-2-Triisostearate and Polyglyceryl-2-Diisostearate making the lipstick more stable. Its dropping point can reach 56-60° C. It is able to keep the lips moist and bright-colored for long time.

In the invention, the weight percentage of said Pigment is 0.5-5%, for example, it can be 0.5%, 1%, 2%, 3%, 4% or 5%, etc.

Preferably, said Pigment comprises any one of Iron Oxide Red, Beta-Carotene, Iron Oxide Yellow, Iron Oxide Black, Black Bean Red, Sorghum Red, Monascus Red, Black Currant Red, Hippophae Rhamnoides Yellow, Buddleia Yellow, Hibiscetin or Gardenia Yellow.

In the invention, the weight percentage of said Lake is 0.5-5%, for example, it can be 0.5%, 1%, 2%, 3%, 4% or 5%, etc.

Preferably, said Lake comprises any one of Amaranth Red Aluminium Lake, Ponceau 4R Altiminium Lake, Erythrosine Aluminium Lake, New Red Aluminium Lake or Tartrazine Aluminium Lake.

In the invention, Pigment can match with Lake optionally in order to make variety of colors.

In the invention, the weight percentage of said Humectant is 10%.

In the invention, Humectant can be general water soluble Humectant or water soluble plant extract. When its weight percentage is 10%, it will bring the best feeling and effect to the skin.

Preferably, said Humectant comprises any one component or the combination of at least two components of Hyaluronic Acid, Chondroitin Sulfate, Aloe, Monocaprylin, Glycerol, Propylene Glycol, 1,2-Pentanediol or Water. Wherein, the typical combinations but not limited to: Monocaprylin, 1,2-Pentanediol and water; Hyaluronic Acid, Chondroitin Sulfate, Aloe and Propylene Glycol.

Preferably, said water-containing lipstick also comprises Antioxidant, Spice and Preservative.

Preferably, the total weight percentage of said Antioxidant, Spice and Preservative is 0.5-2%, for example, it can be 0.5%, 1%, 1.2%, 1.5%, 1.8% or 2%, etc.

In the invention, Antioxidant, Spice and Preservative can be matched in any ratios within the limited percentage range. For example, Antioxidant can be 1%, Spice can be 0.5% and Preservative can be 0.2%.

Preferably, said Antioxidant comprises any one component or the combination of at least two components of Butylated Hydroxytoluene, Tocopherol, Tea Polyphenol, Lecithin, Ethoxyquin, 2,6-Di-Tert-Butylphenol, L-Ascorbyl 6-Stearate or Calcium Diascorbate. Wherein, the typical combinations but not limited to: Butylated Hydroxytoluene, Tocopherol and Lecithin; Tocopherol, Tea Polyphenol, Lecithin and Ethoxyquin.

Preferably, said Spice comprises any one component or the combination of at least two components of Vanillin, Coumarin, Phenethyl Alcohol or Piperonyl Aldehyde. Wherein, the typical combinations but not limited to: Coumarin and Phenethyl Alcohol; Vanillin, Phenethyl Alcohol and Piperonyl Aldehyde.

Preferably, said Preservative comprises any one component or the combination of at least two components of Benzoic Acid, Sodium Benzoate, Potassium Sorbate, Calcium Diacetate or ϵ-Pilylysine. Wherein, the typical combinations but not limited to: Sodium Benzoate and Potassium Sorbate; Benzoic Acid, Potassium Sorbate and Calcium Diacetate.

As the preferable art scheme, The water-containing lipstick provided in the invention comprises following components:

Emollient 20-40%, Hardener 13-15%, Brightener 10-15%, Polyglyceryl-2-Triisostearate 10-15%, Polyglyceryl-2-Diisostearate 10-15%, Pigment 0.5-5%, Lake 0.5%-5% and Humectant 10%, total weight percentage of Antioxidant, Spice and Preservative is 0.5-2%.

The water-containing lipstick of the invention uses such complicated formula, while still comes into good effect. It provides a new idea to lipstick research, and have great significance for lipstick development.

Second, the invention provides the preparation method of making said water-containing lipstick in first part. Said preparation method comprises the following steps:
(1) Use heated and dissolved Hardener to first mix with Emollient and Brightener. Then add Polyglyceryl-2-Diisostearate and Lake to have second mixing and get lipid composition.
(2) Use Polyglyceryl-2-Triisostearate, Pigment, Humectant and any Antioxidant, Preservative and Spice to have third mixing and get hydrous composition.
(3) Put the hydrous composition in Step (2) into lipid composition in Step (1) to have forth mixing, then fill into mould to get said water-containing lipstick.

The preparation method provided in the invention is simple, operation is easy. The ingredients adding sequence is specific during the making process. Otherwise, the uniformity and stability of lipstick will be influenced, dropping point will be lower.

Preferably, said heating temperature in Step (1) is 87-93° C., for example, it can be 87° C., 88° C., 89° C., 90° C., 91° C., 92° C. or 93° C., etc.

Preferably, temperatures of said first and second mixing in Step (1) are 80-85° C., for example, they can be 80° C., 81° C., 82° C., 83° C., 84° C. or 85° C., etc.

Preferably, temperatures of said third and forth mixing in Step (2) and (3) are 80-85° C., for example, they can be 80° C., 81° C., 82° C., 83° C., 84° C. or 85° C., etc. In the invention, before the third mixing, Polyglyceryl-2-Triisostearate, Pigment and Humectant need to be heated to 80-85° C.; then mix and dissolve, stir and emulsify; finally add any Antioxidant, Preservative and Spice.

Preferably, said filling in Step (3) proceeds in certain filling mould.

Preferably, temperature of said filling mould is 80-85° C., for example, it can be 80° C., 81° C., 82° C., 83° C., 84° C. or 85° C., etc.

As the preferable art scheme, the preparation method provided in the invention comprises following steps:
(1) After heating and dissolving Hardener at 87-93° C., first mix it with Emollient and Brightener. Then add Polyglyceryl-2-Diisostearate and Lake to have second mixing and get lipid composition. First and second mixing are processed at 80-85° C.
(2) Mix the Polyglyceryl-2-Triisostearate, Pigment and Humectant at 80-85° C., then stir and emulsify. Then add any Antioxidant, Preservative and Spice to have third mixing and get hydrous composition. Temperature of third mixing is 80-85° C.
(3) Put the hydrous composition in Step (2) into lipid composition in Step (1) to have forth mixing, then fill into mould to get said water-containing lipstick. Temperature of forth mixing is 80-85° C.

Preferably, the composition of forth mixing needs to take vacuum-pumping, then fill into mould to get said water-containing lipstick, in order to reduce influence to overall appearance of water-containing lipstick that made by bubbles.

Preferably, after the forth mixing, the hydrous composition forms hydrous particles and disperse in lipid composition; and such hydrous particles are wrapped by lipid composition. It effectively prevents hydrous lipstick from water loss after filling into the mould.

In comparison with prior art, the invention has the following advantageous effects:

The water-containing lipstick provided in the invention has high level of water content which can reach 10%, can add different kinds of water soluble ingredients, has high stability, dropping point can reach 56-60° C., excellent effect of emulsification, no ultrasonic equipment is required to eliminate bubbles when filling the mould (can use general filling equipment). This lipstick has various colors and high saturation, it can be easily distinguished after using on the lips.

In comparison with current moisturizing lipstick and the ones with other formulas, The water-containing lipstick provided in the invention has both good stability and perfect taste through reasonable match of all complicated components. In the meantime, its moderate colors will show gorgeous and elegant feeling when applying on the lips. It conforms to public aesthetic and has good market prospect.

### BRIEF DESCRIPTION OF THE DRAWINGS

Below are explanations of the invention combining with drawings.

FIG. 1 Process flow diagram of first and second mixing.

FIG. 2 Process flow diagram of third mixing.

FIG. 3 Process flow diagram of forth mixing, vacuum-pumping and filling mould.

### DETAILED DESCRIPTION OF THE INVENTION

Below are the examples further describing the art of invention. The people skilled in the art should be aware that, said examples only help to understand the invention better, but should not be the limit to the invention.

### Example 1

The water-containing lipstick in this example comprises the following components:

Ethylhexyl Palmitate 20%, Octyl 4-Methoxycinnamate 4%, Avobenzone 5%, Carnauba Wax 15%, Cetyl Ethylhexanoate 3%, Squalane Oil 5%, Tri(Tridecyl) Benzene-1,2,4-Tricarboxylate 2%, Polyglyceryl-2-Triisostearate 10%, Polyglyceryl-2-Diisostearate 10%, Iron Oxide Red 4%, Ponceau 4R Altiminium Lake 4.5%, Monocaprylin 1%, Tri(Tridecyl) Benzene-1,2,4-Tricarboxylate 1%, 1,2-Pentanediol 1%, water 7%, Butylated Hydroxytoluene 0.5%, Vanillin 0.5% and Potassium Sorbate 0.5%.

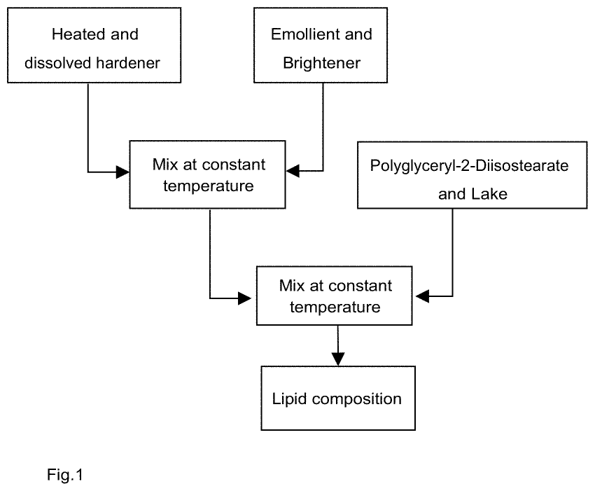

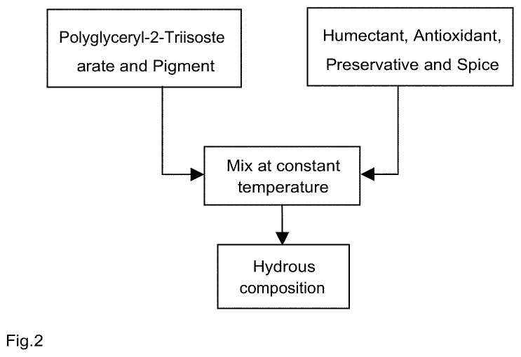

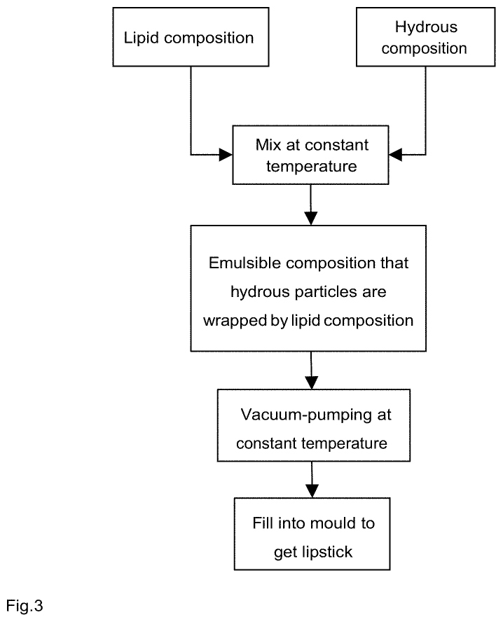

As shown in FIG. 1 to FIG. 3, the preparation method comprises the following steps:
(1) After heating and dissolving Carnauba Wax at 90° C., first mix it with Ethylhexyl Palmitate, Octyl 4-Methoxycinnamate, Avobenzone, Cetyl Ethylhexanoate, Squalane Oil and Tri(Tridecyl) Benzene-1,2,4-Tricarboxylate. First mixing temperature is 82° C. Then add Polyglyceryl-2-Diisostearate and Ponceau 4R Altiminium Lake to have second mixing and get lipid composition. Second mixing temperature is 82° C.
(2) Mix the Polyglyceryl-2-Triisostearate, Iron Oxide Red, Monocaprylin, Tri(Tridecyl) Benzene-1,2,4-Tricarboxylate, 1,2-Pentanediol and water, stir and emulsify them. Then add Butylated Hydroxytoluene, Vanillin and Potassium Sorbate to have third mixing and get hydrous composition. Temperature of third mixing is 82° C.
(3) Put the hydrous composition into lipid composition to have forth mixing at 82° C., then fill into mould to get said water-containing lipstick.

### Example 2

The water-containing lipstick in this example comprises the following components:

Ethylhexyl Palmitate 10%, Carnauba Wax 10%, Bees Wax 5%, Cetyl Ethyl hexanoate 15%, Polyglyceryl-2-Triisostearate 15%, Polyglyceryl-2-Diisostearate 15%, Black Currant Red 4%, Ponceau 4R Altiminium Lake 4%, water 10%, Butylated Hydroxytoluene 1%, Lecithin 0.5%, Coumarin 0.2% and Potassium Sorbate 0.3%.

The preparation method comprises the following steps:
(1) After heating and dissolving Carnauba Wax and Bees Wax at 90° C., first mix them with Ethylhexyl Palmitate and Cetyl Ethylhexanoate at 80° C. Then add Polyglyceryl-2-Diisostearate and Ponceau 4R Altiminium Lake to have second mixing and get lipid composition. Second mixing temperature is 80° C.
(2) Mix the Polyglyceryl-2-Triisostearate, Black Currant Red and water, stir and emulsify them. Then add Butylated Hydroxytoluene, Lecithin, Coumarin and Potassium Sorbate to have third mixing and get hydrous composition. Temperature of third mixing is 80° C.
(3) Put the hydrous composition into lipid composition to have forth mixing at 80° C., then fill into mould to get said water-containing lipstick.

### Example 3

The water-containing lipstick in this example comprises the following components:

Meadowfoam Seed Oil 5%, Ethylhexyl Palmitate 15%, Octyl 4-Methoxycinnamate 9%, Avobenzone 5%, Carnauba Wax 13%, Cetyl Ethylhexanoate 5%, Squalane Oil 5%, Polyglyceryl-2-Triisostearate 11%, Polyglyceryl-2-Diisostearate 11%, Black Bean Red 0.5%, Amaranth Red Aluminium Lake 4.5%, 1,2-Pentanediol and water 8%.

The preparation method comprises the following steps:
(1) After heating and dissolving Carnauba Wax at 90° C., first mix it with Meadowfoam Seed Oil, Ethylhexyl Palmitate, Octyl 4-Methoxycinnamate and, Avobenzone, Cetyl Ethylhexanoate and Squalane Oil at 85° C. Then add Polyglyceryl-2-Diisostearate and Amaranth Red Aluminium Lake to have second mixing and get lipid composition. Second mixing temperature is 85° C.
(2) Mix the Polyglyceryl-2-Triisostearate, Black Bean Red, 1,2-Pentanediol and water, stir and emulsify them to get hydrous composition. The mixing temperature is 80° C.
(3) Put the hydrous composition into lipid composition to have forth mixing, then fill into mould to get said water-containing lipstick.

### Contrast Example 1

The differences between Contrast example 1 and Example 1 are, this example does not comprise Polyglyceryl-2-Triisostearate and the weight percentage of Polyglyceryl-2-Diisostearate is 30%. Besides, the other components and preparation method of making are the same with Example 1, to get water-containing lipstick.

### Contrast Example 2

The differences between Contrast example 2 and Example 1 are, this example does not comprise Polyglyceryl-2-Diisostearate and the weight percentage of Polyglyceryl-2-Triisostearate is 30%. Besides, the other components and preparation method of making are the same with Example 1, to get water-containing lipstick.

### Contrast Example 3

The difference between Contrast example 3 and Example 1 is, this example does not comprise Polyglyceryl-2-Triisostearate and Polyglyceryl-2-Diisostearate. Besides, the other components and preparation method of making are the same with Example 1, to get water-containing lipstick.

### Contrast Example 4

The difference between Contrast example 4 and Example 1 is, this example does not comprise Carnauba Wax 15%. Besides, the other components and preparation method of making are the same with Example 1, to get water-containing lipstick.

### Contrast Example 5

The differences between Contrast example 5 and Example 1 are, the preparation method in this example is: Mix all ingredients directly, stir and emulsify them, finally fill into the mould and get water-containing lipstick.

Functional test: Conducted stability tests (dropping point test: the higher the dropping point is, the better the stability is) and user sensory test (applied the lipsticks to 200 randomly selected females, had overall evaluation through moisturizing, comfort level and coloring; full score 100, lowest score 0, the higher the mark is, the better the lipstick is) with The water-containing lipsticks made in Example 1-3 and Contrast example 1-5. Test results are shown as Table 1 below.

TABLE 1

| Sample | Dropping point (° C.) | Overall score |
|---|---|---|
| Example 1 | 57 | 96 |
| Example 2 | 59 | 90 |
| Example 3 | 56 | 94 |
| Contrast example 1 | 52 | 84 |
| Contrast example 2 | 54 | 86 |
| Contrast example 3 | 51 | 76 |
| Contrast example 4 | 38 | 72 |
| Contrast example 5 | 50 | 67 |

By the comparison between Example 1-3 and Contrast example 1-3, it can be seen the invention takes effect through adding Polyglyceryl-2-Triisostearate and Polyglyceryl-2-Diisostearate together, they coordinate with each other. It will lower the stability of lipstick and influence its taste if any one is missing. When both of them are missing, the taste and stability will drop significantly. By the comparison with Contrast example 4, it can be seen the missing of Carnauba Wax will sharply lower the dropping point, reduce stability and taste. By the comparison with Contrast example 5, it can be seen if directly mixing all ingredients together, the taste of lipstick will be bad that overall score is 67 only, and the stability is low.

Besides, the components of water-containing lipstick in the invention is complicated and mixed by reasonable percentage which provide the best effect. It has relatively high value in practical application, and it also provides a new idea in developing lipstick.

Applicant hereby declares that, the examples described in the invention are used to introduce The water-containing lipstick and its preparation method of making, but not limit to the above preparation method; it doesn't mean the invention must be made by said specific method. The people skilled in the art should be aware that, the following aspects should exist within the scope of protection and disclosure of the invention: any improvement, equivalent substitution of ingredient, addition of auxiliary component, selection of specific approach and etc.

1. A water-containing lipstick, wherein the water-containing lipstick comprises the following components in terms of weight percentage: Emollient 20-40%, Hardener 13-15%, Brightener 10-15%, Polyglyceryl-2-Triisostearate 10-15%, Polyglyceryl-2-Diisostearate 10-15%, Pigment 0.5-5%, Lake 0.5%-5% and Humectant 10%.

2. The water-containing lipstick of claim 1, wherein, said Emollient comprises any one component or the combination of at least two components of Polyoxyethylene Glyceryl Ether Cocoate, Ethylhexyl Palmitate, Olea europaea(Olive) Fruit Oil, Simmondsia chinensis(Jojoba) Seed Oil, Coconut Oil, Cocoamidopropyl Betaine, Octyl Methoxycinnamate, Meadowfoam Seed Oil, Dipolyhydroxystearate, Octyl 4-Methoxycinnamate or Avobenzone.

3. The water-containing lipstick of claim 1, wherein, said Hardener comprises any one component or the combination of at least two components of Bees Wax, Carnauba Wax, Polyethylene or Methyl Cellulose.

4. The water-containing lipstick of claim 1, wherein, said Brightener comprises any one component or the combination of at least two components of Cetyl Ethylhexanoate, Squalane Oil or Tri(Tridecyl) Benzene-1,2,4-Tricarboxylate.

5. The water-containing lipstick of claim 1, wherein, said Pigment comprises any one of Iron Oxide Red, Beta-Carotene, Iron Oxide Yellow, Iron Oxide Black, Black Bean Red, Sorghum Red, Monascus Red, Black Currant Red, Hippophae Rhamnoides Yellow, Buddleia Yellow, Hibiscetin or Gardenia Yellow.
Preferably, said Lake comprises any one of Amaranth Red Aluminium Lake, Ponceau 4R Altiminium Lake, Erythrosine Aluminium Lake, New Red Aluminium Lake or Tartrazine Aluminium Lake.

6. The water-containing lipstick of claim 1, wherein, said Humectant comprises any one component or the combination of at least two components of Hyaluronic Acid, Chondroitin Sulfate, Aloe, Monocaprylin, Glycerol, Propylene Glycol, 1,2-Pentanediol or Water.

7. The water-containing lipstick of claim 1, wherein, said water-containing lipstick also comprises Antioxidant, Spice and Preservative.
Preferably, total weight percentage of said Antioxidant, Spice and Preservative is 0.5-2%.
Preferably, said Antioxidant comprises any one component or the combination of at least two components of Butylated Hydroxytoluene, Tocopherol, Tea Polyphenol, Lecithin, Ethoxyquin, 2,6-Di-Tert-Butylphenol, L-Ascorbyl 6-Stearate or Calcium Diascorbate.
Preferably, said Spice comprises any one component or the combination of at least two components of Vanillin, Coumarin, Phenethyl Alcohol or Piperonyl Aldehyde.
Preferably, said Preservative comprises any one component or the combination of at least two components of Benzoic Acid, Sodium Benzoate, Potassium Sorbate, Calcium Diacetate or ε-Pilylysine.

8. The preparation method of making said water-containing lipstick of claim 1, wherein, said preparation method comprises the following steps.
(1) Use heated and dissolved Hardener to first mix with Emollient and Brightener. Then add Polyglyceryl-2-Diisostearate and Lake to have second mixing and get lipid composition.
(2) Use Polyglyceryl-2-Triisostearate, Pigment, Humectant and any Antioxidant, Preservative and Spice to have third mixing and get hydrous composition.
(3) Put the hydrous composition in Step (2) into lipid composition in Step (1) to have forth mixing, then fill into mould to get said water-containing lipstick.

9. The preparation method of claim 8, wherein, said heating temperature in Step (1) is 87-93° C.
Preferably, temperatures of said first and second mixing in Step (1) are 80-85° C.

10. The preparation method of claim 8, wherein, temperature of said third mixing in Step (2) is 80-85° C.; temperature of said forth mixing in Step (3) is 80-85° C.
Preferably, said filling in Step (3) proceeds in certain filling mould.
Preferably, temperature of said filling mould is 80-85° C.

11. The preparation method of claim 8, wherein, after the forth mixing, the hydrous composition forms hydrous particles and disperse in lipid composition; and such hydrous particles are wrapped by lipid composition.

12. The preparation method of claim 8, wherein, the forth mixing that mixed by hydrous composition and lipid composition in said Step (3) needs to be vacuum-pumping, then fill into mould to get said water-containing lipstick.
